(19) United States
(12) Patent Application Publication
(10) Pub. No.: US 2021/0291164 A1
(43) Pub. Date: Sep. 23, 2021

(54) STOOL SAMPLING CONTAINER

(71) Applicant: FUJIFILM CORPORATION, Tokyo (JP)

(72) Inventor: Kaku IRISAWA, Kanagawa (JP)

(21) Appl. No.: 17/190,441

(22) Filed: Mar. 3, 2021

(30) Foreign Application Priority Data
Mar. 23, 2020 (JP) 2020-051706

(51) Int. Cl. B01L 3/00; A61B 10/00

(52) U.S. Cl. CPC B01L 3/5029; B01L 2300/0809; B01L 2200/025; A61B 10/0038

(57) ABSTRACT

In the inside of a container main body having a first surface into which a stool sampling rod is inserted and a second surface opposed to the first surface, a storage space for storing a storage solution has a wide portion having a width wider than that of the first surface side at an end portion on the second surface side, and the wide portion has a width which enables receiving the nozzle inserted from a first position shifted in the width direction with respect to a coaxial position on an extension line of an axis of the stool sampling rod, and allows the parts of the nozzle and the stool sampling rod to overlap each other in the longitudinal direction.

### CROSS-REFERENCE TO RELATED APPLICATIONS

The present application claims priority under 35 U.S.C. § 119 to Japanese Patent Application No. 2020-051706, filed on Mar. 23, 2020. The above application is hereby expressly incorporated by reference, in its entirety, into the present application.

### BACKGROUND

### 1. Technical Field

The technology of the present disclosure relates to a stool sampling container.

### 2. Description of the Related Art

A stool sampling container used for a fecal occult blood test for detecting colorectal cancer is known. JP2006-029825A discloses a stool sampling container comprising a stool sampling rod for sampling stool and a container main body to which the stool sampling rod is attached. The container main body contains a storage solution in which the stool sampled by the stool sampling rod is suspended.

The stool sampling container disclosed in JP2006-029825A has a substantially rectangular planar shape. In the stool sampling container, the stool sampling rod is inserted from the first surface being located on one side in the longitudinal direction of the container, and a nozzle for extracting a suspension is inserted from the second surface opposed to the first surface in the longitudinal direction.

### SUMMARY

The stool sampling container is required to be miniaturized from the viewpoints of ease of mailing and portability. In the stool sampling container disclosed in JP2006-029825A, the nozzle is inserted from the second surface opposed to the first surface into which the stool sampling rod is inserted, and an insertion position of the nozzle is a coaxial position on the extension line of the axis of the stool sampling rod. Therefore, it is necessary to secure a space between a distal end of the nozzle and the stool sampling rod such that the distal end of the nozzle and the stool sampling rod do not come into contact with each other in a case where the nozzle is inserted, and it is difficult to shorten a length of the stool sampling container in the longitudinal direction.

Further, in addition to the miniaturization, the stool sampling container is required to reduce an amount of the storage solution stored in the container main body. This is because, in a case where the amount of the storage solution is increased, there is a disadvantage that not only the cost of the storage solution increases, but also the amount of the stool to be sampled needs to be increased in order to obtain an appropriate density of the stool in the suspension.

An object of the technology of the present disclosure is to provide a stool sampling container having a small size in the longitudinal direction while suppressing an increase in a storage solution.

A stool sampling container of the present disclosure comprises a stool sampling rod that samples stool, a container main body to which the stool sampling rod is attached and that contains a storage solution in which the stool sampled by the stool sampling rod is suspended, the container main body having the first surface provided at one end in a longitudinal direction parallel to an axial direction of the stool sampling rod in an attached state, and the second surface provided at the other end in the longitudinal direction and opposed to the first surface, in which the stool sampling rod is inserted from the first surface, and a nozzle for extracting the storage solution is inserted from the second surface in parallel to the stool sampling rod, and a storage space that is partitioned by a partition wall inside the container main body and is capable of storing the storage solution in the partition wall, and into which a part including a distal end of the stool sampling rod and a part including a distal end of the nozzle are inserted, in which in a case where a direction orthogonal to the longitudinal direction is defined as a width direction, the storage space has a wide portion formed at an end portion on the second surface side and having a width wider than that of the first surface side, and the wide portion has a width which enables receiving the nozzle inserted from a first position shifted in the width direction with respect to a coaxial position on an extension line of an axis of the stool sampling rod, and allows the parts of the nozzle and the stool sampling rod to overlap each other in the longitudinal direction.

It is preferable that the wide portion has a width which enables maintaining a noncontact state between the nozzle and the stool sampling rod inserted in parallel to each other.

It is preferable that the wide portion has a width which enables receiving the nozzle inserted from the second position different from the first position, the second position being located on the second surface as the first position, and allows the parts of the nozzle inserted from the second position and the stool sampling rod to overlap each other in the longitudinal direction as in a case where the nozzle is inserted from the first position.

It is preferable that the first position is an insertion position at which the nozzle is inserted in a case of extracting the storage solution, and the second position is an insertion position at which the nozzle is inserted in a case of forming an air hole for introducing air into the storage space.

It is preferable that the wide portion extends to both sides about the stool sampling rod in the width direction.

It is preferable that the container main body and the storage space have a symmetrical shape with the stool sampling rod as a center axis in the width direction.

At least a part of the second surface may be formed of a thin membrane of a material different from a material of a main portion of the container main body.

It is preferable that the container main body has an extension portion that is formed around the thin membrane and extends outward from the thin membrane along the longitudinal direction.

It is preferable that at least a part of the second surface is formed of the same material as a main portion of the container main body and has a thin portion relatively thinner than the main portion, and the thin portion has a thickness which is pierced by the nozzle.

According to the technology of the present disclosure, it is possible to provide a stool sampling container having a small size in the longitudinal direction while suppressing an increase in a storage solution.

### BRIEF DESCRIPTION OF THE DRAWINGS

Exemplary embodiments according to the technique of the present disclosure will be described in detail based on the following figures, wherein:

FIG. 1 is a perspective view of a stool sampling container,

FIG. 2 is an exploded perspective view of a stool sampling container,

FIG. 3 is view showing a periphery of the storage space. (A) of FIG. 3 shows a plan view of a container main body viewed from the second surface side, and (B) of FIG. 3 shows a cross-sectional view of the container main body, which is cut along the longitudinal direction.

FIG. 4 is a diagram showing a state in which stool is sampled with a stool sampling rod,

FIG. 5 is a diagram showing a state in which a stool sampling rod in which stool is captured by a capturing portion is returned to a container main body,

FIG. 6 is a diagram showing handling of a stool sampling container in a test apparatus,

FIG. 7 is view showing a periphery of the storage space. (A) of FIG. 7 shows a plan view of a container main body viewed from the second surface side, and (B) of FIG. 7 shows a cross-sectional view of the container main body, which is cut along the longitudinal direction,

FIG. 8 is view showing a periphery of the storage space. (A) of FIG. 8 shows a plan view of a container main body viewed from the second surface side, and FIG. 8B shows a cross-sectional view of the container main body, which is cut along the longitudinal direction, and

FIG. 9 is diagram showing handling of a stool sampling container in a test apparatus. (A) of FIG. 9 shows a state in which a nozzle is inserted into a storage space in a case of forming an air hole in a seal, and (B) of FIG. 9 shows a state in which a nozzle is inserted into a storage space in a case of extracting a suspension.

### DETAILED DESCRIPTION

### First Embodiment

### Configuration of Stool Sampling Container

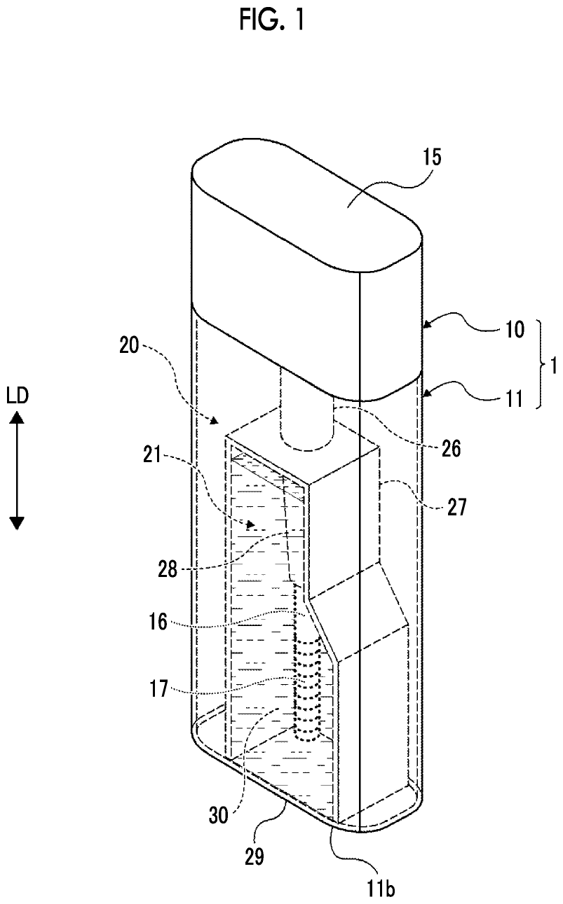

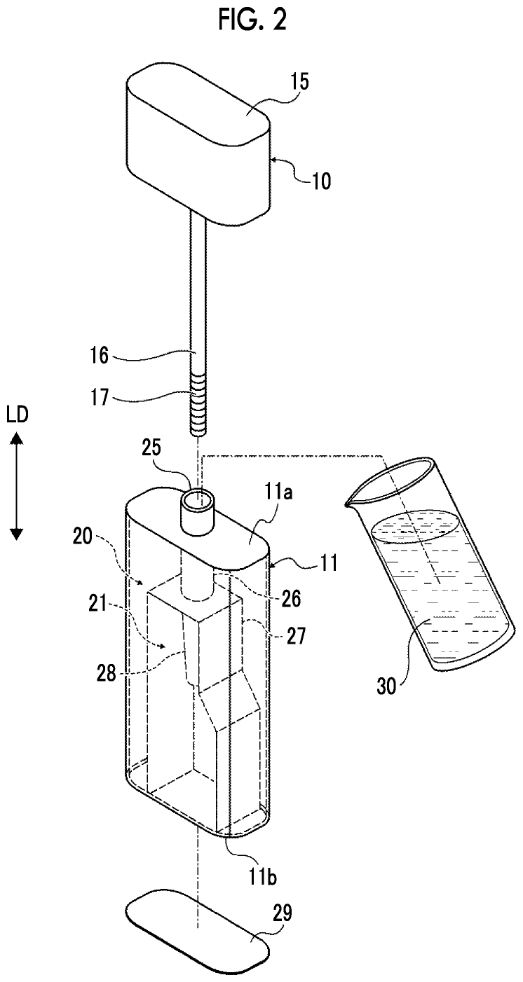

FIG. 1 is a perspective view of a stool sampling container 1 according to the first embodiment of the present disclosure. FIG. 2 is an exploded perspective view of the stool sampling container 1. In the following description, for the sake of convenience, the upper side in FIG. 1 and FIG. 2 will be described as the first surface side of the stool sampling container 1, and the lower side in FIG. 1 and FIG. 2 will be described as the second surface side of the stool sampling container 1.

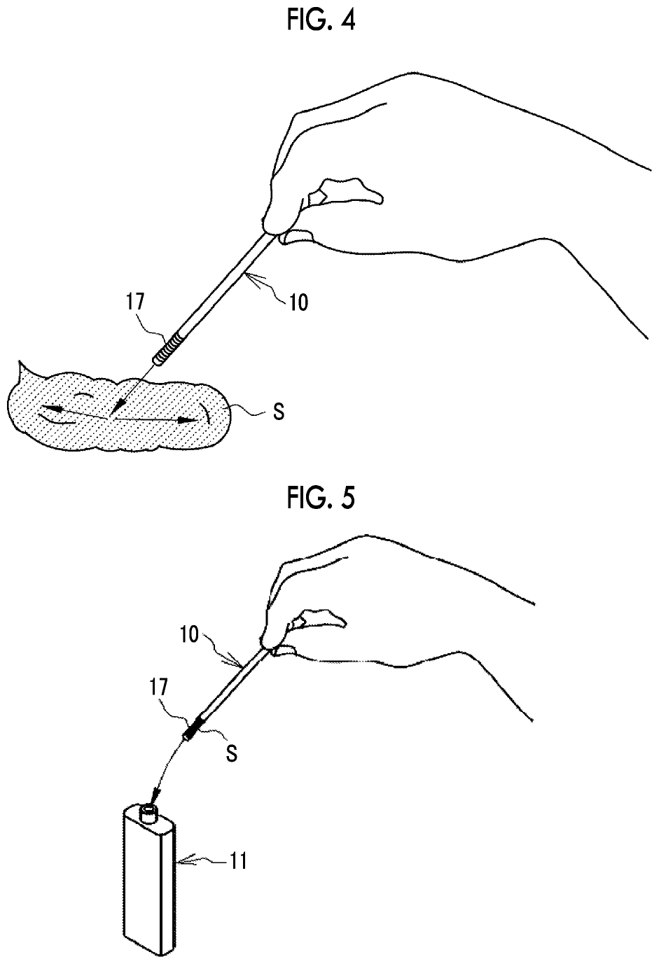

In FIG. 1 and FIG. 2, the stool sampling container 1 is used as an example for a fecal occult blood test for detecting colorectal cancer. The stool sampling container 1 has a substantially rectangular parallelepiped shape that is elongated and flat as a whole. The stool sampling container 1 comprises a stool sampling rod 10 for sampling stool S (refer to FIG. 4 and the like) and a container main body 11 to which the stool sampling rod 10 is attachably and detachably attached.

The stool sampling rod 10 is formed of a colored resin as an example. The stool sampling rod 10 is configured with a cap 15, a rod main body 16, and a capturing portion 17. The cap 15 has a substantially rectangular parallelepiped shape and is gripped by a subject at the time of sampling stool (refer to FIG. 4 and the like). The rod main body 16 has an elongated cylindrical shape extending downward from the cap 15. The capturing portion 17 is formed at a distal end of the rod main body 16. A plurality of grooves for efficiently capturing the stool S are formed in the capturing portion 17.

In the first embodiment, the container main body 11 is configured with a main body portion 11d and a seal 29 described later. The main body portion 11d is the main portion of the container main body 11, and the seal 29 forms the second surface 11b of the container main body 11. The main body portion 11d is formed of a transparent resin such that the inside can be visually recognized from the outside. The transparent resin includes, for example, an acrylonitrile-butadiene-styrene (ABS) resin, polyethylene, polypropylene, and the like.

The container main body 11 is attached with the stool sampling rod 10 and stores a storage solution 30 in which the stool sampled by the stool sampling rod 10 is suspended. The stool sampling rod 10 is inserted into the container main body 11 in a posture in which the axial direction is parallel to a longitudinal direction LD. In the container main body 11, one end in the longitudinal direction LD parallel to the axial direction (the axial direction of the rod main body 16 in the first embodiment) of the attached stool sampling rod 10 has the first surface 11a, and the other end has the second surface 11b opposed to the first surface 11a. From the first surface 11a, the stool sampling rod 10 is inserted, and from the second surface 11b, as described later, a nozzle 60 for extracting the storage solution 30 is inserted.

A cylindrical shaped plug portion 25 which is press-fitted to the cap 15 to prevent liquid leakage from the container main body 11 is formed on the first surface 11a (refer to FIG. 2) of the container main body 11. The plug portion 25 functions as a stool sampling rod insertion portion.

The container main body 11 is partitioned by a partition wall 27 inside an outer peripheral wall 11c, and has a storage space 21 capable of storing the storage solution 30. Between the outer peripheral wall 11c and the storage space 21, there is a buffer space 20 for relaxing an impact and the like from the outside.

In the buffer space 20, an insertion portion 26 is provided between the first surface 11a and the storage space 21. The insertion portion 26 is a tubular body having a cylindrical shape. In the insertion portion 26, in a case where the stool sampling rod 10 is inserted from the plug portion 25 of the first surface 11a into the container main body 11, the rod main body 16 including the capturing portion 17 which has passed through the plug portion 25 is inserted.

On the second surface 11b side of the insertion portion 26, a scraping-off portion 28 for scraping off excess stool adhering to the capturing portion 17 is provided. The scraping-off portion 28 is a conical tubular body. The inner diameter of the scraping-off portion 28 is gradually narrowed from the insertion portion 26 toward the second surface 11b side to the same extent as the outer diameter of the capturing portion 17. The scraping-off portion 28 is integrally molded with, for example, the partition wall 27.

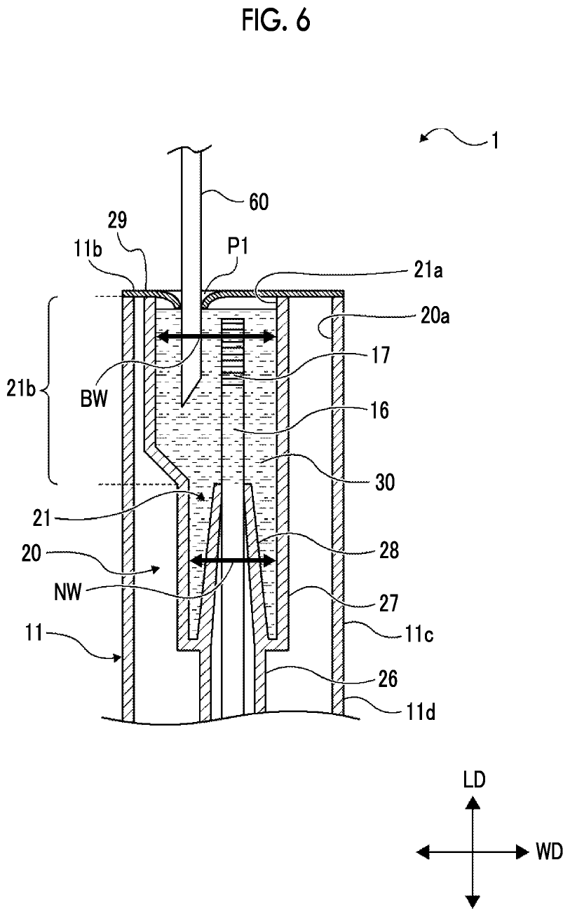

The storage solution 30 is stored in the storage space 21. The storage solution 30 contains water as the main component. In the state shown in FIG. 1 in which the stool sampling rod 10 is attached to the container main body 11, the entire capturing portion 17 enters the storage space 21, and the distal end of the capturing portion 17 reaches a vicinity of the second surface 11b in the longitudinal direction LD of the container main body 11. That is, a part including the capturing portion 17 which is the distal end of the stool sampling rod 10 is inserted into the storage space 21. Then, about 90% of the storage space 21 is filled with the storage solution 30. Therefore, the entire capturing portion 17 is immersed in the storage solution 30. The stool S captured by the capturing portion 17 is suspended in the storage solution 30. Hereinafter, the storage solution 30 in which the stool S is suspended is referred to as a suspension 30S (refer to FIG. 6).

In the main body portion 11d of the container main body 11, an opening 20a of the buffer space 20 and an opening 21a of the storage space 21 are formed at an end portion on the second surface 11b side. The opening 20a and the opening 21a are covered by the seal 29. The seal 29 consists of a thin membrane of metal or resin, and is attached to the end portion of the main body portion 11d on the second surface 11b side by, for example, heat welding. As a result, the seal 29 functions as the second surface 11b of the container main body 11.

The opening 21a is provided to introduce the nozzle 60 (refer to FIG. 6) for extracting the suspension 30S into the storage space 21. The seal 29 is punctured by the nozzle 60 in a case of extracting the suspension 30S for the fecal occult blood test (refer to FIG. 6). In this way, a part including the distal end of the nozzle 60 is inserted into the storage space 21.

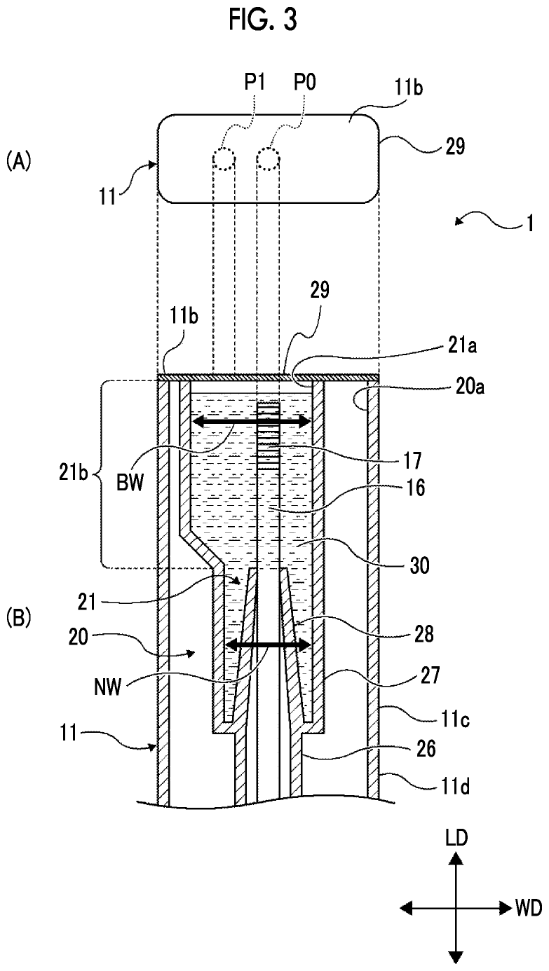

FIG. 3 is view showing a periphery of the storage space 21. (A) of FIG. 3 shows a plan view of a container main body 11 viewed from the second surface 11b side, and (B) of FIG. 3 shows a cross-sectional view of the container main body 11, which is along the longitudinal direction. It should be noted that (B) of FIG. 3 shows a state in which the container main body 11 is turned upside down from that in FIG. 1. That is, in (B) of FIG. 3, the second surface 11b faces upward. In addition, in (B) of FIG. 3, the rod main body 16 of the stool sampling rod 10 is shown not as a cross-sectional view but as a side view. Further, as shown in (A) of FIG. 3 and (B) of FIG. 3, in the container main body 11, a direction orthogonal to the longitudinal direction LD is defined as a width direction WD.

As shown in (A) of FIG. 3 and (B) of FIG. 3, in the width direction WD, the storage space 21 has a wide portion 21b having a width wider than that of the first surface 11a side at the end portion on the second surface 11b side. A width BW of the wide portion 21b is an interval between the partition walls 27 in the width direction WD. In the storage space 21, an interval between the partition walls 27 on a side closer to the first surface 11a than the wide portion 21b is a width NW. The width BW is wider than the width NW. The wide portion 21b can receive the nozzle 60 inserted from the first position P1 shifted in the width direction WD with respect to a coaxial position P0 on an extension line of the axis (the rod main body 16 in the first embodiment) of the stool sampling rod 10, and has the width BW allowing parts of the nozzle 60 and the stool sampling rod 10 to overlap each other in the longitudinal direction LD. The coaxial position P0 is a position at which the second surface 11b and the extension line of the axis of the stool sampling rod 10 intersect.

As described above, the nozzle 60 is inserted from the first position P1 shifted in the width direction WD with respect to the coaxial position P0 of the stool sampling rod 10 in the surface of the second surface 11b. Since the second surface 11b is configured with the seal 29, in a case where the nozzle 60 is inserted, the first position P1 of the seal 29 is punctured by the nozzle 60.

In addition, in the first embodiment, the wide portion 21b has a width BW which enables maintaining a noncontact state between the nozzle 60 and the stool sampling rod 10 inserted in parallel with each other (refer to FIG. 3 and FIG. 6). In other words, the wide portion 21b has a width BW which enables receiving both the nozzle 60 and the stool sampling rod 10 with an interval therebetween.

### Procedure of Examination

Next, a procedure from stool sampling by the stool sampling container 1 having the above configuration to the fecal occult blood test will be described with reference to FIG. 4 to FIG. 6. First, a subject excretes stool S into a toilet bowl. Then, the stool sampling rod 10 is removed from the container main body 11, and as shown in FIG. 4, the capturing portion 17 of the stool sampling rod 10 is brought into contact with the stool S, and the capturing portion 17 traces a surface of the stool S. Since the capturing portion 17 has a plurality of grooves, it is possible to easily capture a necessary and sufficient amount of stool S for the fecal occult blood test.

Subsequently, as shown in FIG. 5, the subject returns the stool sampling rod 10 in which the stool S is captured by the capturing portion 17 to the container main body 11 and puts it in the state shown in FIG. 1. At this time, the capturing portion 17 is inserted through the insertion portion 26, passes through the scraping-off portion 28, and reaches the storage space 21. In a case where the capturing portion 17 passes through the scraping-off portion 28, the excess stool S among the stools S captured by the capturing portion 17 is removed.

After stool sampling, the stool sampling container 1 is transported to a test facility where the fecal occult blood test is performed. During this transportation, the storage solution 30 in the storage space 21 is agitated. Then, the stool S captured by the capturing portion 17 is diffused into the storage solution 30, and the suspension 30S suitable for a fecal occult blood test is generated.

As shown in FIG. 6, in the test facility, the stool sampling container 1 is set on a mounting portion of the test apparatus (not shown) in a state of being upside down from that in FIG. 1. Then, in the test apparatus, the nozzle 60 pierces into the seal 29 and is inserted into the storage space 21 of the container main body 11. In the test apparatus, a positional relationship between the nozzle 60 and the mounting portion is determined, and a disposition position of the nozzle 60 is set at a position at which the nozzle 60 is inserted from the first position P1 on the second surface 11b of the container main body 11 mounted on the mounting portion.

In the test apparatus, after the nozzle 60 is inserted into the storage space 21 from the first position P1, the suspension 30S in the storage space 21 is extracted by the nozzle 60 for the fecal occult blood test.

### Action Effect

As described above, in the stool sampling container 1 of the present disclosure, the container main body 11 is provided with a storage space 21, and in a case where the direction orthogonal to the longitudinal direction LD is defined as the width direction WD, the storage space 21 has the wide portion 21b having a width wider than that of the first surface 11a side at the end portion on the second surface 11b side. Then, the wide portion 21b can receive the nozzle 60 inserted from the first position P1 shifted in the width direction WD with respect to the coaxial position P0 on the extension line of the axis of the stool sampling rod 10. The wide portion 21b has the width BW that allows the parts of the nozzle 60 and the stool sampling rod 10 to overlap each other in the longitudinal direction LD.

As described above, in the stool sampling container 1 of the present disclosure, since the nozzle 60 and the stool sampling rod 10 are allowed to overlap each other in the longitudinal direction LD in the storage space 21, in comparison with a conventional stool sampling container in which the nozzle is inserted at a coaxial position with the axis of the stool sampling rod, the lengths of the storage space 21 and the container main body 11 having the storage space 21 inside can be reduced in the longitudinal direction LD. That is, in the prior art, since the stool sampling rod and the nozzle are at coaxial position in the storage space, the length of the storage space in the longitudinal direction LD is required to be long enough to secure a space for disposing the nozzle on the extension line of the stool sampling rod. On the other hand, in the stool sampling container 1 of the present disclosure, since the wide portion 21b is provided in the storage space 21 to allow the nozzle 60 and the stool sampling rod 10 to overlap each other in the longitudinal direction LD, the lengths of the storage space 21 and the container main body 11 can be shortened in the longitudinal direction LD.

In addition, in the storage space 21, only the width BW of the wide portion 21b into which the nozzle 60 is inserted is widened, and the width NW of a region other than the wide portion 21b remains narrower than the width BW, so that the increase in the amount of the storage solution 30 stored in the storage space 21 can be suppressed.

Therefore, according to the technology of the present disclosure, it is possible to provide the stool sampling container 1 having a small size in the longitudinal direction while suppressing an increase in the amount of the storage solution 30.

In addition, in the stool sampling container 1 of the present disclosure, since the stool sampling rod 10 and the nozzles 60 are inserted in parallel to each other along the longitudinal direction LD, for example, in comparison with a case where the stool sampling rod 10 and the nozzle 60 are inserted in a direction in which the axial directions intersect with each other, it is easy to change the shape of the container main body 11 into a simple rectangular parallelepiped shape with no irregularities in the outer periphery. Therefore, the storage and handling of the stool sampling container 1 is easier than that of the stool sampling container having irregularities in the outer periphery.

In addition, the wide portion 21b have the width BW which enables maintaining a noncontact state between the nozzle 60 and the stool sampling rod 10 inserted into the storage space 21 in parallel with each other. Therefore, since the nozzle 60 and the capturing portion 17 do not come into contact with each other, it is possible to prevent the stool that is not suspended in the storage solution 30 and remains adhered to the capturing portion 17 from adhering to the nozzle 60. As a result, it is possible to suppress the density of the stool in the suspension 30S extracted by the nozzle 60 from exceeding an appropriate value. Further, since the noncontact state between the nozzle 60 and the stool sampling rod 10 is maintained, an abnormal stop of the test apparatus as a result that the nozzle 60 does not reach a target position by the contact between the nozzle 60 and the stool sampling rod 10 can be suppressed, or the possibility of breakage by the contact between the nozzle 60 and the stool sampling rod 10 can be reduced.

In addition, the second surface 11b of the container main body 11 is formed by the seal 29 which is an example of a thin membrane of a material different from the material of the main body portion 11d which is the main portion of the container main body 11. Therefore, for example, the main body portion 11d of the container main body 11 can be configured so as not to be deformed carelessly by using a material having an intensity higher than that of the second surface 11b. On the other hand, the second surface 11b can be configured by selecting a material having a certain liquid tightness while giving priority to ease of insertion of the nozzle 60. In this way, by discriminating the material of the main portion of the container main body 11 and the material of the second surface 11b, it is possible to appropriately select the material according to a performance required for the container main body 11.

In the first embodiment, the example in which the entire surface of the second surface lib is formed by the seal 29 is described, but a part of the second surface lib may be formed by the seal 29.

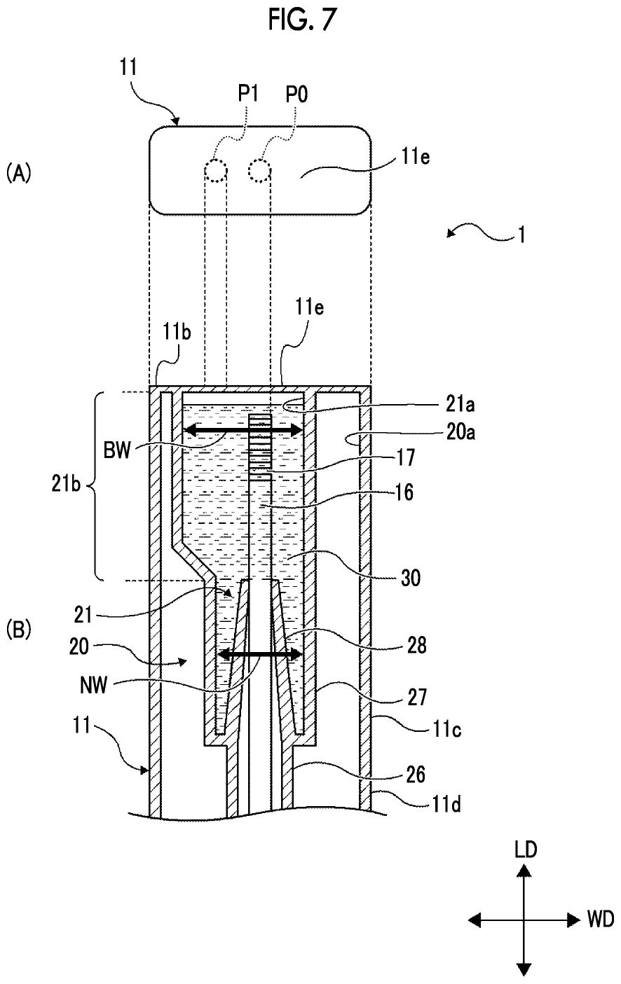

In addition, as shown in FIG. 7, the second surface 11b of the container main body 11 may be formed of the same material as the main body portion 11d of the container main body 11. In this case, in the container main body 11, a portion that functions as the second surface lib is preferably a thin portion 11e that is relatively thinner than the main body portion 11d. More specifically, the thickness of the thin portion 11e is a thickness which can be pierced by the nozzle 60. By forming the entire container main body 11 with the same material, it may be possible to reduce a material cost as compared with a case where another material is used.

In addition, the thin portion 11e may be integrally molded with the main body portion 11d. With such a configuration, a manufacturing cost can be reduced since an assembly process of attaching the second surface lib to the main body portion 11d is eliminated.

### Second Embodiment

### Configuration of Stool Sampling Container

A stool sampling container 2 of the second embodiment is mainly formed by changing the shape of the storage space 21 from the stool sampling container 1 of the first embodiment. Therefore, the stool sampling container 2 of the second embodiment will be described mainly in terms of differences from the stool sampling container 1 of the first embodiment, and the description of the same portion will be omitted as appropriate.

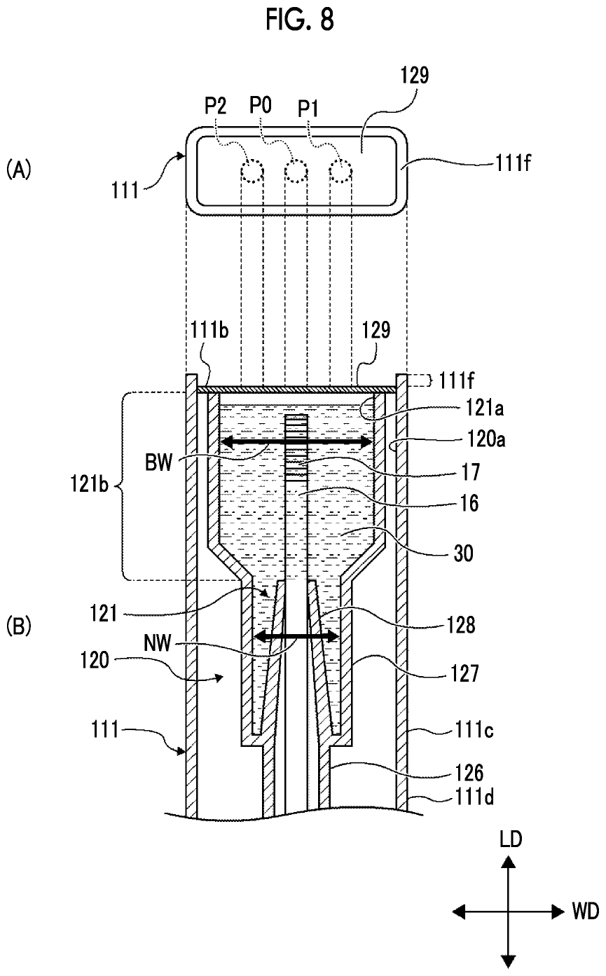

As shown in (A) of FIG. 8 and FIG. 8B, also in the container main body 111 of the stool sampling container 2 of the second embodiment, like the container main body 11 of the first embodiment, the first surface is provided at one end in the longitudinal direction LD parallel to the axial direction of the attached stool sampling rod 10, and the second surface 111b opposed to the first surface is provided at the other end. The stool sampling rod 10 is inserted from the first surface, and the nozzle 60 for extracting the storage solution 30 is inserted from the second surface 111b.

Similar to the container main body 11 of the first embodiment, the container main body 111 is partitioned by a partition wall 127 inside the outer peripheral wall 111c, and has a storage space 121 in which the storage solution 30 is stored. Between the outer peripheral wall 111c and the storage space 121, there is a buffer space 120 similar to the buffer space 20 of the first embodiment. The storage space 121 of the container main body 111 has a different shape from the storage space 21 of the first embodiment.

Similar to the buffer space 20 of the first embodiment, the buffer space 120 is formed with an insertion portion 126 into which the rod main body 16 including the capturing portion 17 is inserted. The insertion portion 126 is a tubular body having a cylindrical shape.

On the second surface 111b side of the insertion portion 126, a scraping-off portion 128 for scraping off excess stool adhering to the capturing portion 17 is provided. The scraping-off portion 128 is a conical tubular body. The inner diameter of the scraping-off portion 128 is gradually narrowed from the insertion portion 126 toward the second surface 111b side to the same extent as the outer diameter of the capturing portion 17. The scraping-off portion 28 is integrally molded with, for example, the partition wall 127.

In the main body portion 111d of the container main body 111, an opening 120a of the buffer space 120 and an opening 121a of the storage space 121 are formed at an end portion on the second surface 111b side. The opening 120a and the opening 121a are covered by the seal 129. The seal 129 consists of a thin membrane of metal or resin, and is attached to the end portion of the main body portion 111d on the second surface 111b side by, for example, heat welding. As a result, the seal 129 functions as the second surface 111b of the container main body 111. The container main body 111 comprises an extension portion 111f extending outward from the seal 129 in the longitudinal direction LD around the seal 129.

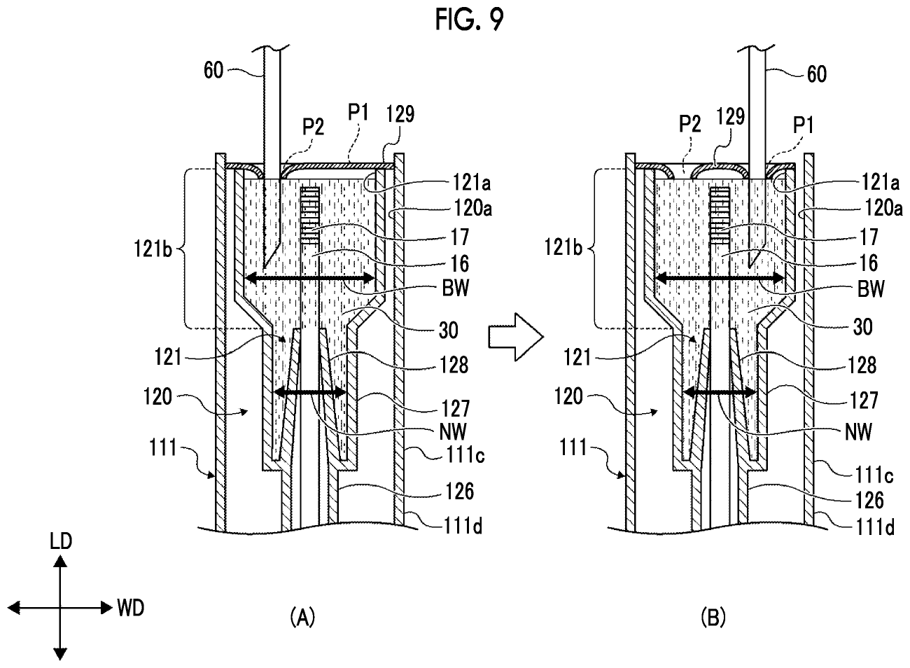

The opening 121a is provided to introduce the nozzle 60 (refer to (A) of FIG. 9 and (B) of FIG. 9) for extracting the suspension 30S into the storage space 121. The seal 129 is punctured by the nozzle 60 in a case of extracting the suspension 30S for the fecal occult blood test. In this way, a part including the distal end of the nozzle 60 is inserted into the storage space 121.

In the width direction WD, the storage space 121 has a wide portion 121b having a width wider than that of the first surface side at the end portion on the second surface 111b side. A width BW of the wide portion 121b is an interval between the partition walls 127 in the width direction WD. In the storage space 121, an interval between the partition walls 127 on a side closer to the first surface than the wide portion 121b is the width NW. The width BW is wider than the width NW. The wide portion 121b can receive the nozzle 60 inserted from the first position P1 and the second position P2 shifted in the width direction WD with respect to a coaxial position P0 on an extension line of the axis of the stool sampling rod 10, and has the width BW allowing parts of the nozzle 60 and the stool sampling rod 10 to overlap each other in the longitudinal direction LD. The coaxial position P0 is a position at which the second surface 111b and the extension line of the axis of the stool sampling rod 10 intersect.

The wide portion 121b extends to both sides about the rod main body 16 of the stool sampling rod 10 in the width direction WD. In addition, the container main body 111 and the storage space 121 have a symmetrical shape with the rod main body 16 the stool sampling rod 10 as a center axis in the width direction WD.

As described above, the nozzle 60 is inserted from the first position P1 and the second position P2 shifted in the width direction WD with respect to the coaxial position P0 of the stool sampling rod 10 in the surface of the second surface 111b. Since the second surface 111b is configured with the seal 129, in a case where the nozzle 60 is inserted, the first position P1 and the second position P2 of the seal 129 is punctured by the nozzle 60.

In addition, in the second embodiment, the wide portion 121b has a width BW which enables maintaining a noncontact state between the nozzle 60 and the stool sampling rod 10 inserted in parallel with each other (refer to (A) of FIG. 9 and (B) of FIG. 9). In other words, the wide portion 121b has a width BW which enables receiving both the nozzle 60 and the stool sampling rod 10 with an interval therebetween.

### Procedure of Examination

Next, a procedure from stool sampling by the stool sampling container 2 having the above configuration to the fecal occult blood test will be described. First, a subject excretes stool S into a toilet bowl. Then, the stool sampling rod 10 is removed from the container main body 111, and as shown in FIG. 4, the capturing portion 17 of the stool sampling rod 10 is brought into contact with the stool S, and the capturing portion 17 traces a surface of the stool S.

Subsequently, as shown in FIG. 5, the subject returns the stool sampling rod 10 in which the stool S is captured by the capturing portion 17 to the container main body 111. At this time, the capturing portion 17 is inserted through the insertion portion 126, passes through the scraping-off portion 128, and reaches the storage space 121. In a case where the capturing portion 17 passes through the scraping-off portion 128, the excess stool S among the stools S captured by the capturing portion 17 is removed.

After stool sampling, the stool sampling container 2 is transported to a test facility where the fecal occult blood test is performed. During this transportation, the storage solution 30 in the storage space 121 is agitated. Then, the stool S captured by the capturing portion 17 is diffused into the storage solution 30, and the suspension 30S suitable for a fecal occult blood test is generated.

As shown in FIG. 9, in the test facility, the stool sampling container 2 is set on a mounting portion of the test apparatus (not shown) in a state of being upside down from that in FIG. 1. Then, in the test apparatus, the nozzle 60 pierces into the seal 129 and is inserted into the storage space 121 of the container main body 111. In the test apparatus, the positional relationship between the nozzle 60 and the mounting portion is determined. The nozzle 60 is configured to be movable between a position at which the nozzle 60 is inserted into the first position P1 and a position at which the nozzle 60 is inserted into the second position P2 on the second surface 111b of the container main body 111 mounted on the mounting portion.

In the test apparatus, first, the nozzle 60 is inserted into the storage space 121 from the second position P2, and an air hole for the storage space 121 is formed. Next, after the nozzle 60 is inserted into the storage space 121 from the first position P1, the suspension 30S in the storage space 121 is extracted by the nozzle 60 for the fecal occult blood test.

### Action Effect

As described above, in the stool sampling container 2 of the present disclosure, the container main body 111 is provided with a storage space 121, and in a case where the direction orthogonal to the longitudinal direction LD is defined as the width direction WD, the storage space 121 has the wide portion 121b having a width wider than that of the first surface 11a side at the end portion on the second surface 111b side. Then, the wide portion 121b can receive the nozzle 60 inserted from the first position P1 and the second position P2 shifted in the width direction WD with respect to the coaxial position P0 on the extension line of the axis of the stool sampling rod 10. The wide portion 121b has the width BW that allows the parts of the nozzle 60 and the stool sampling rod 10 to overlap each other in the longitudinal direction LD.

As described above, in the stool sampling container 2 of the present disclosure, since the nozzle 60 and the stool sampling rod 10 are allowed to overlap each other in the longitudinal direction LD in the storage space 121, in comparison with a conventional stool sampling container in which the nozzle is inserted at a coaxial position with the axis of the stool sampling rod, the lengths of the storage space 121 and the container main body 111 having the storage space 121 inside can be reduced in the longitudinal direction LD. That is, in the prior art, since the stool sampling rod and the nozzle are at coaxial position in the storage space, the length of the storage space in the longitudinal direction LD is required to be long enough to secure a space for disposing the nozzle on the extension line of the stool sampling rod. On the other hand, in the stool sampling container 2 of the present disclosure, since the wide portion 121b is provided in the storage space 121 to allow the nozzle 60 and the stool sampling rod 10 to overlap each other in the longitudinal direction LD, the length of the storage space 121 and the container main body 111 can be shortened in the longitudinal direction LD.

In addition, in the storage space 121, only the width BW of the wide portion 121b into which the nozzle 60 is inserted is widened, and the width NW of a region other than the wide portion 121b remains narrower than the width BW, so that the increase in the amount of the storage solution 30 stored in the storage space 121 can be suppressed.

Therefore, according to the technology of the present disclosure, it is possible to provide the stool sampling container 2 having a small size in the longitudinal direction while suppressing an increase in the amount of the storage solution 30.

In addition, the wide portion 121b may have the width BW which enables maintaining a noncontact state between the nozzle 60 and the stool sampling rod inserted into the storage space 121 in parallel with each other. Therefore, since the nozzle 60 and the capturing portion 17 do not come into contact with each other, it is possible to prevent the stool that is not suspended in the storage solution 30 and remains adhered to the capturing portion 17 from adhering to the nozzle 60. As a result, it is possible to suppress the density of the stool in the suspension 30S extracted by the nozzle 60 from exceeding an appropriate value. Further, since the noncontact state between the nozzle 60 and the stool sampling rod 10 is maintained, the nozzle 60 is brought into contact with the stool sampling rod 10 to prevent the nozzle 60 from reaching a target position. As a result, an abnormal stop of the test apparatus can be suppressed, or the possibility of breakage due to the contact between the nozzle 60 and the stool sampling rod 10 can be reduced.

In addition, the wide portion 121b may have the width BW which enables receiving the nozzle 60 inserted from the second position P2 different from the first position P1, the second position P2 being located on the second surface 111b as the first position P1, and allows the parts of the nozzle 60 inserted from the second position P2 and the stool sampling rod 10 to overlap each other in the longitudinal direction LD as in a case where the nozzle 60 is inserted from the first position P1. Therefore, since the nozzle 60 can be inserted from two different places in the storage space 121, the convenience of the stool sampling container 2 can be improved.

Among the two nozzle insertion positions, the first position P1 is an insertion position at which the nozzle 60 is inserted in a case where the suspension 30S is extracted, and the second position P2 is an insertion position at which the nozzle 60 is inserted in a case where an air hole for introducing air into the storage space 121 is formed. Therefore, since the air hole is formed in the seal 129 sealing the storage space 121 and the suspension 30S in the storage space 121 can be extracted, the inside of the storage space 121 does not have negative pressure in a case of extracting the suspension 30S, and the suspension 30S can be stably extracted.

In addition, the wide portion 121b extends to both sides about the rod main body 16 of the stool sampling rod 10 in the width direction WD. Therefore, in the case where the nozzle 60 can be inserted into two different positions as described above, it is not necessary to widen the width from the rod main body 16 to one side, so that the stool sampling container 2 can be easily miniaturized.

In addition, the container main body 111 and the storage space 121 have a symmetrical shape with the rod main body 16 the stool sampling rod 10 as a center axis in the width direction WD. Therefore, in a case where the stool sampling container 2 is mounted into the test apparatus in which the nozzle 60 are inserted into two different positions as described above, the stool sampling container 2 can be mounted without worrying horizontal directions, so that convenience can be improved.

In addition, the container main body 111 comprises the extension portion 111f formed around the seal 129 and extending outward from the seal 129 along the longitudinal direction LD. Therefore, it is possible to reduce a risk that the seal 129 is rubbed and peeled off in a case where the stool sampling container 2 is used, transported, or the like.

### Modification Example

The technology of the present disclosure can be appropriately combined with the above-described various embodiments and various modification examples. In addition, the technology of the present disclosure is not limited to the above-described embodiments, and it is needless to say that various configurations can be adopted without departing from the gist of the present invention.

The description content and the illustrated content described above are detailed descriptions of portions related to the technology of the present disclosure, and are merely an example of the technology of the present disclosure. For example, the above description of the configurations, functions, actions, and effects is an example of the configurations, functions, actions, and effects of the portions according to the technology of the present disclosure. Therefore, it is needless to say that in the description content and the illustrated content described above, unnecessary portions may be deleted and new elements may be added to or replaced within the scope not deviating from the gist of the technology of the present disclosure. In addition, in order to avoid confusion and to facilitate understanding portions according to the technology of the present disclosure, the description of the common general technical knowledge and the like, which need not be specifically explained in order to enable implementation of the technology of the present disclosure, is omitted from the description content and the illustrated content described above.

All of the documents, patent applications, and technical standards described herein are incorporated herein by reference to the same extent as a case where the individual documents, patent applications, and technical standards are specifically and individually indicated to be incorporated by reference.

What is claimed is:

1. A stool sampling container comprising:
a stool sampling rod that samples stool;
a container main body to which the stool sampling rod is attached and that contains a storage solution in which the stool sampled by the stool sampling rod is suspended, the container main body having a first surface provided at one end in a longitudinal direction parallel to an axial direction of the stool sampling rod in an attached state, and a second surface provided at the other end in the longitudinal direction and opposed to the first surface, in which the stool sampling rod is inserted from the first surface, and a nozzle for extracting the storage solution is inserted from the second surface in parallel to the stool sampling rod; and
a storage space that is partitioned by a partition wall inside the container main body and is capable of storing the storage solution in the partition wall, and into which a part including a distal end of the stool sampling rod and a part including a distal end of the nozzle are inserted,
wherein in a case where a direction orthogonal to the longitudinal direction is defined as a width direction, the storage space has a wide portion formed at an end portion on the second surface side and having a width wider than that of the first surface side, and
the wide portion has a width which enables receiving the nozzle inserted from a first position shifted in the width direction with respect to a coaxial position on an extension line of an axis of the stool sampling rod, and allows the parts of the nozzle and the stool sampling rod to overlap each other in the longitudinal direction.

2. The stool sampling container according to claim 1,
wherein the wide portion has a width which enables maintaining a noncontact state between the nozzle and the stool sampling rod inserted in parallel to each other.

3. The stool sampling container according to claim 1,
wherein the wide portion has a width which enables receiving the nozzle inserted from a second position different from the first position, the second position being located on the second surface as the first position, and allows the parts of the nozzle inserted from the second position and the stool sampling rod to overlap each other in the longitudinal direction as in a case where the nozzle is inserted from the first position.

4. The stool sampling container according to claim 3,
wherein the first position is an insertion position at which the nozzle is inserted in a case of extracting the storage solution, and
the second position is an insertion position at which the nozzle is inserted in a case of forming an air hole for introducing air into the storage space.

5. The stool sampling container according to claim 3,
wherein the wide portion extends to both sides about the stool sampling rod in the width direction.

6. The stool sampling container according to claim 5,
wherein the container main body and the storage space have a symmetrical shape with the stool sampling rod as a center axis in the width direction.

7. The stool sampling container according to claim 1,
wherein at least a part of the second surface is formed of a thin membrane of a material different from a material of a main portion of the container main body.

8. The stool sampling container according to claim 7,
wherein the container main body has an extension portion that is formed around the thin membrane and extends outward from the thin membrane along the longitudinal direction.

9. The stool sampling container according to claim 1,
wherein at least a part of the second surface is formed of the same material as a main portion of the container main body and has a thin portion relatively thinner than the main portion, and
the thin portion has a thickness which is pierced by the nozzle.
